(19) United States
(12) Patent Application Publication
(10) Pub. No.: US 2006/0045908 A1
(43) Pub. Date: Mar. 2, 2006

(54) Ready-made supplementary feed for horses

(75) Inventor: Hans-Hinnrich Moss, Scharnebeck (DE)

Correspondence Address: NIXON PEABODY, LLP, 401 9TH STREET, NW, SUITE 900, WASHINGTON, DC, 20004-2128, US

(73) Assignee: Scharnebecker Muehle Dienstleistung und Handelsgesellschaft mbH, Scharnebeck (DE)

(21) Appl. No.: 11/206,152

(22) Filed: Aug. 18, 2005

(30) Foreign Application Priority Data
Aug. 26, 2004 (DE) 10 2004 041 400.9

(51) Int. Cl. A61K 36/899; A61K 36/185; A23K 1/165

(52) U.S. Cl. 424/442; 424/750; 424/776

(57) ABSTRACT

Ready-made supplementary feed for horses that is also suited to prophylaxis or therapy of the muscle metabolism condition PSSM, contains 5-25 weight % rice meal and/or rice bran, 12-30 weight % supplying vegetable fat constituents, 20-40 weight % of green meal, 20-40 weight % of sugar beet seeds. The constituents supplying vegetable fat contain approximately 10 weight % of at least one of black and striped sunflower seed kernels, 2 weight % of soy bean oil, 3 weight % of linseed cake and 3 weight % of linseed oil. The feed can also contain approximately 5 weight % dried pulp, 5 weight % apple residue, 5 weight % of at least one of brewer's yeast and soy grits.

### BACKGROUND OF THE INVENTION

### Field of the Invention

The present invention relates to ready-made supplementary feed for horses, including feed, for example, manufactured in pelletized form, or as so-called muesli, on an industrial scale and commercially available in larger to large packaged units. This ready-made supplementary feed is also described as complementary feed, since it is usually fed to horses which are simultaneously being feeding on grass (grazing), hay and straw (litter). Customary other complementary feed is mineral feed.

### SUMMARY OF THE INVENTION

A primary object of the present invention is to provide a ready-made supplementary feed for horses of the above-mentioned type, which is additionally suited to prophylaxis or therapy of the muscle metabolism condition “Polysaccharide-Storage-Myopathy” (PSSM).

This object is obtained by a ready-made supplementary feed containing 5-25 weight % of at least one of rice meal and rice bran, 12-30 weight % of constituents supplying vegetable fat, 20-40 weight % of green meal, and 20-40 weight % of sugar beet seeds.

### DETAILED DESCRIPTION OF THE INVENTION

The concept of inventive ready-made supplementary feed for horses also takes into consideration the assumption that such feed should be gluten-free as much as possible.

Extensive testing has proven that the inventive combination of feed constituents of the inventive ready-made supplementary feed is a high-value substitute for ready-made supplementary feed for horses commercially available to date; at the same time, it serves as prophylaxis or therapy for the muscle metabolism condition “Polysaccharide-Storage-Myopathy” (PSSM). It is also emphasized, in particular, that, in the combination, the bitter taste, inherent to rice meal or rice bran, and which per se essentially hampers intake by horses, is overplayed.

The inventive ready-made supplementary feed for horses contains 5-25% of rice meal and/or rice bran, 12-30% of constituents supplying vegetable fat, 20-40% of green meal, and 20-40% of sugar beet seeds, all of indicated percentages relating to the weight of the ready-made supplementary feed. Preferably, the feed contains 5-15 wt. % rice meal and/or rice bran, 15-25 wt. % of constituents supplying vegetable fat, 15-35 wt. % of green meal and 15-35 wt. % of sugar beet seeds. Particularly preferred is a content of 8-12 wt. % rice meal and/or rice bran, 16-20 wt. % constituents supplying vegetable fat, 23-30 wt. % green meal and 22-28 wt. % sugar beet seeds, in particular 10 wt. % of rice meal and/or rice bran, 18 wt. % of constituents supplying vegetable fat, 27 wt. % of green meal and 25 wt. % of sugar beet seeds.

The constituents supplying vegetable fat preferably comprise approximately 10 wt. % of sunflower seed kernels (black and/or striped), 2 wt. % soy bean oil, 3 wt. % linseed cake and 3 wt. % linseed oil.

The inventive ready-made supplementary feed, in particular, preferably, in addition, contains approximately 5 wt. % dried pulp, 5 wt. % apple residue, 5 wt. % brewer's yeast and/or 5 wt. % soy grits.

What is claimed is:

1. Ready-made supplementary feed for horses, also suited to prophylaxis or therapy of the muscle metabolism condition PSSM, containing:
5-25 weight % of at least one of rice meal and rice bran,
12-30 weight % of constituents supplying vegetable fat,
20-40 weight % of green meal, and
20-40 weight % of sugar beet seeds.

2. The ready-made supplementary feed as claimed in claim 1, containing:
5-25 weight % of at least one of rice meal and rice bran,
15-25 weight % of constituents supplying vegetable fat,
15-35 weight % of green meal, and
15-35 weight % of sugar beet seeds.

3. The ready-made supplementary feed as claimed in claim 1, containing:
8-12 weight % of at least one of rice meal and rice bran,
16-20 weight % of constituents supplying vegetable fat,
23-30 weight % of green meal, and
22-28% sugar beet seeds.

4. The ready-made supplementary feed as claimed claim 1, containing:
10 weight % of at least one of rice meal and rice bran,
18 weight % of constituents supplying vegetable fat,
27 weight % green meal, and
25 weight % of sugar beet seeds.

5. The ready-made supplementary feed as claimed in claim 4, further comprising approximately 5 weight % dried pulp, 5 weight % apple residue, 5 weight % of at least one of brewer's yeast and soy grits.

6. The ready-made supplementary feed as claimed in claim 1, further comprising approximately 5 weight % dried pulp, 5 weight % apple residue, 5 weight % of at least one of brewer's yeast and soy grits.

7. The ready-made supplementary feed as claimed in claim 6, wherein the constituents supplying vegetable fat comprise approximately 10 weight % of at least one of black and striped sunflower seed kernels, 2 weight % of soy bean oil, 3 weight % of linseed cake and 3 weight % of linseed oil.

8. The ready-made supplementary feed as claimed in claim 5, wherein the constituents supplying vegetable fat comprise approximately 10 weight % of at least one of black and striped sunflower seed kernels, 2 weight % of soy bean oil, 3 weight % of linseed cake and 3 weight % of linseed oil.

9. The ready-made supplementary feed as claimed in claim 4, wherein the constituents supplying vegetable fat comprise approximately 10 weight % of at least one of black and striped sunflower seed kernels, 2 weight % of soy bean oil, 3 weight % of linseed cake and 3 weight % of linseed oil.

10. The ready-made supplementary feed as claimed in claim 1, wherein the constituents supplying vegetable fat comprise approximately 10 weight % of at least one of black and striped sunflower seed kernels, 2 weight % of soy bean oil, 3 weight % of linseed cake and 3 weight % of linseed oil.
